(19) United States
(12) Patent Application Publication
(10) Pub. No.: US 2009/0005323 A1
(43) Pub. Date: Jan. 1, 2009

(54) Cathepsin K Inhibitors and Obesity

(76) Inventor: Michael David Percival, Montreal West (CA)

Correspondence Address: MERCK AND CO., INC, P O BOX 2000, RAHWAY, NJ, 07065-0907, US

(21) Appl. No.: 11/795,447

(22) PCT Filed: Jan. 17, 2006
(86) PCT No.: PCT/CA2006/000054

§ 371 Date: Jul. 17, 2007

Related U.S. Application Data
(60) Provisional application No. 60/644,926, filed on Jan. 19, 2005.

(51) Int. Cl. A61K 31/70; A61K 31/164; A61K 31/495; A61K 31/445; A61K 31/423; C07D 241/04; A61P 3/04; C07D 211/06; C07C 233/01; A61K 31/421; A61K 31/4355; A61K 31/5375

(52) U.S. Cl. 514/23; 514/613; 514/252.12; 514/331; 514/377; 514/282; 514/231.2; 514/379; 564/123; 544/358; 546/246

(57) ABSTRACT

This invention relates to the treatment of obesity, the treatment of obesity related disorders, prevention of weight gam, prevention of weight regain or for weight maintenance by the use of a cathepsin K inhibitor as active ingredient, alone or in conjunction with other anti-obesity agents The invention also relates to the pharmaceutical compositions comprising cathepsin K inhibitor as active ingredient, pharmaceutically acceptable carriers or excipients, and optionally one or more anti-obesity agents.

### BACKGROUND OF THE INVENTION

This invention relates to the treatment of obesity, treatment of an obesity related disorder, prevention of weight gain, prevention of weight regain and use for weight maintenance by the administration of a cathepsin K inhibitor, either as a single agent or in combination with other agents.

Cathepsin K expression is upregulated in white adipose tissue in several mouse models of obesity, whereas cathepsin K mRNA levels are not affected in other non-adipose tissues. Cathepsin K expression is induced during differentiation of mouse 3T3-F442A cells into adipocytes. Furthermore, in lean and obese male humans, a significant correlation between cathepsin K gene expression in adipose tissue and body mass index is observed (Chiellini, C. Cell Physiol. 2003, 195:309-21). These data show that a relationship exists between cathepsin K and adipogenesis and obesity.

Cathepsin K may play a key role in adipogenesis and the development of obesity, and cathepsin K inhibitors may be useful for the treatment of obesity and related disorders such as overeating and bulimia, hypertension, type 1 diabetes, type 2 diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The present data show that genetic ablation of cathepsin K in female mice which were fed a high fat diet for 12 weeks results in a reduction in weight gain and adiposity compared to wild-type littermate female rice.

### SUMMARY OF THE INVENTION

The instant invention relates to a method of treating of obesity, treating an obesity related disorder, preventing weight gain, preventing weight regain or using for weight maintenance by the administration of a cathepsin K inhibitor, either as a single agent or in combination with other agents.

### DETAILED DESCRIPTION OF THE INVENTION

The instant invention relates to a method of treating of obesity, treating an obesity related disorder, preventing weight gain, preventing weight regain or using for weight maintenance with a cathepsin K inhibitor, either as a single agent or in combination with other agents. In an embodiment of the invention, the cathepsin K inhibitor is a compound of formula I:

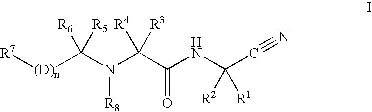

and the pharmaceutically acceptable salts, esters and solvates thereof wherein:

wherein R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;

R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;

or R¹ and R² can be taken together with the carbon atom to which they are attached to form a C₃₋₈ cycloalkyl or heterocyclyl ring wherein said ring system is optionally substituted with one or two substituents independently selected from C₁₋₆ alkyl, hydroxyalkyl, haloalkyl, or halo;

R³ is hydrogen, C₁₋₆ alkyl or C_{2-≢}alkenyl wherein said alkyl and alkenyl groups are optionally substituted with C₃₋₆ cycloalkyl or one to six halo;

R⁴ is hydrogen, C₁₋₆ alkyl or C₂₋₆ alkenyl wherein said alkyl and alkenyl groups are optionally substituted with C₃₋₆ cycloalkyl or one to six halo;

or R³ and R⁴ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted with one or two substitutents independently selected from C₁₋₆ alkyl, halo, hydroxyalkyl, hydroxy, alkoxy or keto;

R⁵ is selected from hydrogen or C₁₋₆ alkyl substituted with 1-6 halo;

R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with one, two, or three substituents independently selected from halo, C₁₋₆ alkyl, C₁₋₆ haloalkyl, C₃₋₆ cycloalkyl, haloalkoxy, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹), cyano, or aryl which is optionally substituted with —SO₂R¹²;

each D is independently C₁₋₃ alkyl, C₂₋₃ alkenyl, C₂₋₃ alkynyl, aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from C₁₋₆ alkyl, haloalkyl, halo, keto, alkoxy, —SR⁹, —SR¹², —OR⁹, —OR¹², N(R¹²)₂, —SO₂R⁹, or —SO₂R¹⁰;

R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR⁹, —OR¹⁰, —C(O)R¹⁰, —R¹⁰C(O)R⁹, —C(O)R⁹, —C(O)N(R^{a})(R^{b}), —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SOₘN(R^{c})(R^{d}), —SOₘCH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R⁹, —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}), C(R^{a})(R^{b})C(O)N(R^{a})(R^{b}), —B(OH)₂, —OCH₂O— or 4,4,5,5-tetramethyl-1,3,2-dioxaborolan-2-yl; wherein said groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SR¹², —SR⁹, —SOₘN(R^{c})(R^{d}), —SOₘN(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —C(O)(R^{a})(R^{b}), —N(R¹⁰)C(R¹⁰)(R¹¹)(R⁹), —N(R¹⁰)CO(R⁹), —NH(CH₂)₂OH, —NHC(O)OR¹⁰, —Si(CH₃)₃, heterocycyl, aryl, or heteroaryl;

R⁸ is hydrogen or C₁₋₆ alkyl;

or R⁴ and R⁸ or can be taken together with any of the atoms to which they may be attached or are between them to form a 4-10 membered heterocyclyl ring system wherein said ring system, which may be monocyclic or bicyclic, is optionally substituted with one or two substituents independently selected from C₁₋₆ alkyl, halo, hydroxyalkyl, hydroxy, keto, —OR¹⁰, —SR¹⁰ or —N(R¹⁰)₂;

R⁹ is selected from the group consisting of hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with one, two, or three substituents independently selected from halo, alkoxy or —SO₂R¹²;

R¹⁰ is hydrogen or C₁₋₆ alkyl

R¹¹ is hydrogen or C₁₋₆ alkyl;

R¹² is hydrogen or C₁₋₆ alkyl which is optionally substituted with one, two, or three substituents independently selected from halo, alkoxy, cyano, —NR¹⁰ or —SR¹⁰;

R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;

R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;

or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with one or two substituents independently selected from C₁₋₆ alkyl and halo;

R^{c} is hydrogen or C₁₋₆ alkyl which is optionally substituted with one, two, or three substituents independently selected from halo or —OR⁹;

R^{d} is hydrogen or C₁₋₆ alkyl which is optionally substituted with one, two, or three substituents independently selected from halo or —OR⁹;

or R^{c} and R^{d} can be taken together with the nitrogen atom to which they are attached or are between them to form a C₃₋₈ heterocyclyl ring which is optionally substituted with one or two substituents independently selected from C₁₋₆ alkyl, halo hydroxyalkyl, hydroxy, alkoxy or keto;

n is independently selected from an integer from zero to three;

each m is independently selected from an integer from zero to two;

and the pharmaceutically acceptable salts, stereoisomers and N-oxide derivatives thereof.

Nonlimiting examples of compound of formula I include:
N¹-(1-cyanocyclopropyl)-4-fluoro- N²-{(1S)-2,2,2-trifluoro-1-[4′-(methylsulfonyl)-1,1′-biphenyl-4-yl]ethyl}-L-leucinamide;
N¹-(1-cyanocyclopropyl)-4-fluoro-N²-{(1S)-2,2,2-trifluoro-1-[4′-(methylsulfinyl)-1,1′-biphenyl-4-yl]ethyl}-L-leucinamide;
N¹(cyanomethyl)-N²{(1S)-2,2,2-trifluoro-1-[4′-(methylsulfonyl)-1,1′-biphenyl-4-yl]ethyl}-L-leucinamide;
N²{(1S)-1-[4′-(aminosulfonyl)-1,1′-biphenyl-4-yl]-2,2,2-trifluoroethyl}-N¹(cyanomethyl)-L-leucinamide;
N¹(1-cyanocyclopropyl)-N²-{(1S)-2,2-difluoro-1-[4′-(methylsulfonyl)biphenyl-4-yl]ethyl}-4-fluoro-L-leucinamide;
   and the pharmaceutically acceptable salts thereof.

Methods of preparation for the above compounds are described in International Publication WO 03/075836, which published on Sep. 18, 2003.

Nonlimiting examples of cathepsin K inhibitors of the present invention also include:
N-(1-{[(cyanomethyl)amino]carbonyl}cyclohexyl)-4-(4-propylpiperazin-1-yl)benzamide;
N-(1-{[(cyanomethyl)amino]carbonyl}cyclohexyl)-4-[1-(2-methoxyethyl)piperidin-4-yl]benzamide;
   and the pharmaceutically acceptable salts thereof. Methods of preparation for these compounds are described in International Publication WO 99/24460, which published on May 20, 1999.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having one to ten carbon atoms unless otherwise specified. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on.

“Alkoxy” or “alkyloxy” represents an alkyl group as defined above, unless otherwise indicated, wherein said alkyl group is attached through an oxygen bridge.

The term “cycloalkyl” or “carbocycle” shall mean cyclic rings of alkanes of three to eight total carbon atoms, unless otherwise indicated, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “cycloalkenyl” shall mean cyclic rings of 3 to 10 carbon atoms, unless otherwise specified, containing at least 1 carbon to carbon double bond (i.e., cyclopropenyl, cyclobutenyl, cyclopenentyl, cyclohexenyl, cycloheptenyl or cycloocentyl).

The term “alkynyl” refers to a hydrocarbon radical straight or branched, containing from 2 to 10 carbon atoms, unless otherwise specified, containing at least 1 carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃) CH₂CH(CH₃)Ph, and so on.

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, and tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” means an alkyl radical as defined above, unless otherwise specified, that is substituted with one to five, preferably one to three halogen. Representative examples include, but are not limited to trifluoromethyl, dichloroethyl, and the like.

The term “haloalkoxy” represents a radical —OR where R is alkyl as defined above that is substituted with one to five, preferably one to three halogen. Representative examples include, but are not limited to trifluoromethyloxy, dichloroethyloxy, and the like.

The term “arylalkyl” includes an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, and chlorophenylethyl. Examples of alkylaryl include, but are not limited to, toluyl, ethylphenyl, and propylphenyl.

The term “heteroarylalkyl” as used herein, shall refer to a system that includes a heteroaryl portion, where heteroaryl is as defined above, and contains an alkyl portion. Examples of heteroarylalkyl include, but are not limited to, thienylmethyl, thienylethyl, thienylpropyl, pyridylmethyl, pyridylethyl and imidazoylmethyl.

The term “cycloalkylalkyl” includes an alkyl portion where alkyl is as defined above and also includes an cycloalkyl portion where cycloalkyl is as defined above. Examples of cycloalkylalkyl include, but are not limited to, cyclopropylmethyl, cyclopentylmethyl, cyclohexylmethyl, cyclopropylethyl, and the like.

The term “hydroxyalkyl” means a linear monovalent hydrocarbon raidcal of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocycle” or “heterocyclyl” as used herein is intended to mean a 5- to 10-membered nonaromatic ring, unless otherwise specified, containing from 1 to 4 heteroatoms selected from the group consisting of O, N, S, SO, or SO₂ and includes bicyclic groups. “Heterocyclyl” therefore includes, but is not limited to the following: piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also emcompassed by this definition.

The present invention also includes N-oxide derivatives and protected derivatives of compounds of Formula I. For example, when compounds of Formula I contain an oxidizable nitrogen atom, the nitrogen atom can beconverted to an N-oxide by methods well known in the art. Also whencompounds of Formula I contain groups such as hydroxy, carboxy, thiol or anygroup containing a nitrogen atom(s), these groups can be protected with a suitable protecting groups. A comprehensive list of suitable protective groups can be found in T.W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The cathepsin K inhibitor compounds of the present invention can be administered in such oral dosage forms as tablets, capsules (each of which includes sustained release or timed release formulations), pills, powders, granules, elixers, tinctures, suspensions, syrups and emulsions. Likewise, they may also be administered in intravenous (bolus or infusion), intraperitoneal, topical (e.g., ocular eyedrop), subcutaneous, intramuscular or transdermal (e.g., patch) form, all using forms well known to those of ordinary skill in the pharmaceutical arts.

The dosage regimen utilizing the cathepsin K inhibitor compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the cathepsin K inhibitor compounds, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, cathepsin K inhibitor compounds may be administered in a single daily dose, or the total daily dosage may be administered in divided doses of two, three or four times daily. Furthermore, preferred cathepsin K inhibitor compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regimen.

In the methods of the present invention, the compounds herein described can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

Exemplifying the invention is a pharmaceutical composition comprising a cathepsin K inhibitor, an anti-obesity agent and a pharmaceutically acceptable carrier. Further illustrating the invention is the use of an cathepsin K inhibitor, an anti-obesity agent and a pharmaceutically acceptable carrier for the preparation of a medicament useful in the treatment of obesity. In an embodiment of the invention, the anti-obesity agent is: PYY, PYY₃₋₃₆, a PYY agonist, 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; aminorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; topiramate; zonisamide; or a combination thereof.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The cathepsin K inhibitor compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Cathepsin K inhibitor compounds may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

According to a further aspect of the present invention, it may be desirable to treat any of the aforementioned conditions with a combination of a cathepsin K inhibitor and one or more other pharmacologically active agents suitable for the treatment of obesity. The cathepsin K inhibitor and the other pharmacologically active agent(s) may be administered to a patient simultaneously, sequentially or in combination. For example, the present compound may be employed directly in combination with the other active agent(s), or it may be administered prior, concurrent or subsequent to the administration of the other active agent(s). In general, the currently available dosage forms of the known therapeutic agents for use in such combinations will be suitable.

The cathepsin K inhibitors of the present invention can be used for the treatment of obesity or obesity related disorders comprising administering a therapeutically effective amount of a cathepsin K inhibitor to a patient in need of such treatment. A further aspect of this invention involves a method for preventing or reducing the risk of developing obesity, comprising administering a prophylactically effective amount of a cathepsin K inhibitor to a patient in need of such treatment. In an embodiment of the invention, obesity related disorders include, but are not limited to: diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, metabolic syndrome, fatty liver; cerebral infarction, cerebral thrombosis, transient ischemic attack, orthopedic disorders, arthritis deformans, lumbodynia, emmeniopathy, infertility, overeating; bulimia, elevated plasma insulin concentrations, hyperlipidemia, endometrial, cancer, breast cancer, prostate cancer, colon cancer, osteoarthritis, cholelithiasis, gallstones, coronary heart disease, abnormal heart rhythms, heart arrythmias, myocardial infarction, polycystic ovarian disease, craniopharyngioma, the Prader-Willi Syndrome, Frohlich's syndrome, GH-deficient subjects, normal variant short stature, Turner's syndrome, metabolic syndrome, or acute lymphoblastic leukemia.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, metabolic syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Prevention of weight regain, prevention of weight gain and use for weight maintenance refer to the administration of the compounds or combinations of the present invention to reduce or maintain the body weight of a subject at risk of obesity. One outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders.

“Treatment” (of obesity and obesity-related disorders) refers to the administration of the compounds or combinations of the present invention to reduce food intake, to reduce body weight, or to maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce food intake, to reduce body weight, or to maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, metabolic syndrome, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, and increased anesthetic risk. The combinations of the present invention are also useful for reducing the risk of secondary outcomes of obesity, such as reducing the risk of left ventricular hypertrophy. The combinations of the present invention are also useful to treat Alzheimer's disease.

The cathepsin K inhibitors are also useful for treating or preventing obesity and obesity-related disorders in cats and dogs. As such, the term “mammal” includes companion animals such as cats and dogs.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

A therapeutically effective amount of a cathepsin K inhibitor can be used for the preparation of a medicament useful for treating or preventing any of the medical conditions described herein, in dosage amounts described herein. Additionally, the medicament may be useful for preventing or reducing the risk of developing obesity, halting or slowing the progression of obesity once it has become clinically manifest. The medicament comprised of a compound of Formula I may also be prepared with one or more additional active agents, such as those described herein.

In a broad embodiment, any suitable additional active agent or agents, including but not limited to anti-obesity agents, may be used in combination with the compound of formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities.

The present invention also provides a method for the treatment or prevention of obesity, which method comprises administration to a patient in need of such treatment an amount of a compound of the present invention and an amount of another agent useful in treating obesity and obesity-related conditions, such that together they give effective relief.

Cathepsin K inhibitors may be used in combination with other drugs that are used in the treatment/prevention/suppression or amelioration of the diseases or conditions for which cathepsin K inhibitors are useful. Such other drugs may be administered, by a route and in an amount commonly used therefor, contemporaneously or sequentially with a cathepsin K inhibitor. When a cathepsin K inhibitor is used contemporaneously with one or more other drugs, a pharmaceutical composition containing such other drugs in addition to the cathepsin K inhibitor is preferred. Accordingly, the pharmaceutical compositions of the present invention include those that also contain one or more other active ingredients, in addition to a cathepsin K inhibitor.

Examples of other active ingredients that may be combined with a cathepsin K inhibitor for the treatment or prevention of obesity and/or diabetes, either administered separately or in the same pharmaceutical compositions, include, but are not limited to:

(a) insulin sensitizers including (i) PPARγ antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like), and compounds disclosed in WO 97/10813, WO 97/27857, WO 97/28115, WO 97/28137, and WO 97/27847; (iii) biguanides such as metformin and phenformin;

(b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂);

(c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide;

(d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like;

(e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activater receptor α agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics;

(f) PPARα agonists such as beclofibrate, benzafibrate, ciprofibrate, clofibrate, etofibrate, fenofibrate, and gemfibrozil; and other fibric acid derivatives, such as Atromid®, Lopid® and Tricor®, and the like, and PPARα agonists as described in WO 97/36579 by Glaxo;

(g) PPARδ agonists, such as those disclosed in WO97/28149;

(h) PPAR α/δ agonists, such as muraglitazar, and the compounds disclosed in U.S. Pat. No. 6,414,002; and

(i) anti-obesity agents, such as (1) growth hormone secretagogues, growth hormone secretagogue receptor agonists/antagonists, such as NN703, hexarelin, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB₁ receptor antagonists or inverse agonists, such as rimonabant (Sanofi Synthelabo), AMT-251, and SR-14778 and SR 141716A (Sanofi Synthelabo), SLV-319 (Solvay), BAY 65-2520 (Bayer), and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, W002/076949, and WO 03/007887; and EPO Application No. EP-658546, EP-656354, EP-576357; (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A, and such as those disclosed in U.S. Pat. No. 5,705,515, and U.S. Pat. No. 5,451,677 and PCT Patent Publications WO94/18161, WO95/29159, WO97/46556, WO98/04526 and WO98/32753, WO 01/74782, and WO 02/32897; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-58708 1X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175 (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027, and Japanese Patent Application Nos. JP 13226269, and JP 2004-139909; (11) melanin-concentrating hormone 2 receptor (MCH2R) agonist/antagonists; (12) orexin-1 receptor antagonists, such as SB-334867-A, and those disclosed in PCT Patent Application Nos. WO 01/96302, WO 01/68609, WO 02/51232, and WO 02/51838; (13) serotonin reuptake inhibitors such as fluoxetine, paroxetine, and sertraline, and those disclosed in U.S. Pat. No. 6,365,633, and PCT Patent Application Nos. WO 01/27060 and WO 01/162341; (14) melanocortin agonists, such as Melanotan II or those described in WO 99/64002 and WO 00/74679; (15) other Mc4r (melanocortin 4 receptor) agonists, such as CHIR86036 (Chiron), ME-10142, and ME-10145 (Melacure), CHIR86036 (Chiron); PT-141, and PT-14 (Palatin), and those disclosed in: U.S. Pat. Nos. 6,410,548; 6,294,534; 6,350,760; 6,458,790; 6,472,398; and 6,376,509; US Patent Publication No. US2004/009751, and PCT Application Nos. WO 99/64002; WO 00/74679; WO 01/70708; WO 01/70337; WO 01/74844; WO 01/91752; WO 01/991752; WO 02/059095; WO 02/059107; WO 02/059108; WO 02/059117; WO 02/068387; WO 02/068388; WO 02/067869; WO 02/11715; WO 02/12166; WO 02/12178; WO 03/007949; and WO 03/009847; (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR146131, and those discribed in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm.(Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); (25) β-hydroxy steroid dehydrogenase-1 inhibitors (β-HSD-1); 26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 Aug; 8(8): 461-75); (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron), and those disclosed in PCT Application Nos. WO 94/09134, WO 98/22128, and WO 99/43813; (35) monoamine reuptake inhibitors, such as sibutramine, and those disclosed in U.S. Pat. Nos. 4,746,680, 4,806,570, and 5,436,272, U.S. Patent Publication No. 2002/0006964 and PCT Application Nos. WO 01/27068, and WO 01/62341; (36) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in PCT Patent Application No. WO 99/00123; (37) thyroid hormone β agonists, such as KB-2611 (KaroBioBMS), and those disclosed in PCT Application No. WO 02/15845, and Japanese Patent Application No. JP 2000256190; (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444; and the compounds disclosed in U.S. Pat. No. 6,699,871, specifically the dihydrogenphosphate salt of 7-[(3R)-3-amino-4-(2,4,5-trifluorophenyl)butanoyl]-3-trifluoromethyl)-5,6,7,8-tetrahydro-1,2,4-triazolo[4,3-a]pyrazine, which is incorporated herein by reference; and International Patent Application Nos. WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); and (50) Topiramate (Topimax®); and (50) peptide YY, PYY 3-36, peptide YY analogs, derivatives, and fragments such as BIM-43073D, BIM-43004C (Olitvak, D.A. et al., Dig. Dis. Sci. 44(3):643-48 (1999)), and those disclosed in U.S. Pat. No. 5,026,685, U.S. Pat. No. 5,604,203, U.S. Pat. No. 5,574,010, U.S. Pat. No. 5,696,093, U.S. Pat. No. 5,936,092, U.S. Pat. No. 6,046,162, U.S. Pat. No. 6,046,167, U.S. Pat. No. 6,093,692, U.S. Pat. No. 6,225,445, U.S. Pat. No. 5,604,203, U.S. Pat. No. 4,002,531, U.S. Pat. No. 4,179,337, U.S. Pat. No. 5,122,614, U.S. Pat. No. 5,349,052, U.S. Pat. No. 5,552,520, U.S. Pat. No. 6,127,355, WO 95/06058, WO 98/32466, WO 03/026591, WO 03/057235, WO 03/027637, and WO 2004/066966, which are incorporated herein by reference; (51) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (52) Neuropeptide Y4 (NPY4) agonists such as pancreatic peptide (PP) as described in Batterham et al., J. Clin. Endocrinol. Metab. 88:3989-3992 (2003), and other Y4 agonists such as 1229U91; (54) cyclo-oxygenase-2 inhibitors such as etoricoxib, celecoxib, valdecoxib, parecoxib, lumiracoxib, BMS347070, tiracoxib or JTE522, ABT963, CS502 and GW406381, and pharmaceutically acceptable salts thereof; (55) Neuropeptide Y1 (NPY1) antagonists such as BIBP3226, J-115814, BIBO 3304, LY-357897, CP-671906, GI-264879A and those disclosed in U.S. Pat. No. 6,001,836; and PCT Application Nos. WO 96/14307, WO 01/23387, WO 99/51600, WO 01/85690, WO 01/85098, WO 01/85173, and WO 01/89528; (56) Opioid antagonists such as nalmefene (Revex®), 3-methoxynaltrexone, naloxone, naltrexone, and those disclosed in: PCT Application No. WO 00/21509; (57) 11β HSD-1 (11-beta hydroxy steroid dehydrogenase type 1) inhibitor such as BVT 3498, BVT 2733, and those disclosed in WO 01/90091, WO 01/90090, WO 01/90092, and U.S. patent No. U.S. Pat. No. 6,730,690 and US Publication No. US 2004-0133011, which are incorporated by reference herein in their entirety; and (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; and (88) zonisamide.

Specific NPY5 antagonists of use in combination with a compound of the present invention are selected from the group consisting of:

(1) 3-oxo-N-(5-phenyl-2-pyrazinyl)-spiro[isobenzofuran-1(3H),4′-piperidine]-1′-carboxamide,

(2) 3-oxo-N-(7-trifluoromethylpyrido[3,2-b]pyridin-2-yl)spiro-[isobenzofuran-1(3H),4′ -piperidine]-1′-carboxamide,

(3) N-[5-(3-fluorophenyl)-2-pyrimidinyl]-3-oxospiro-[isobenzofuran-1(3H),4′-piperidine]-1′-carboxamide,

(4) trans-3′-oxo-N-(5-phenyl-2-pyrimidinyl)spiro[cyclohexane-1,1′ (3′H)-isobenzofuran]-4-carboxamide,

(5) trans-3′-oxo-N-[1-(3-quinolyl)-4-imidazolyl]spiro[cyclohexane-1,1′(3′H)-isobenzofuran]-4-carboxamide,

(6) trans-3-oxo-N-(5-phenyl-2-pyrazinyl)spiro[4-azaiso-benzofuran-1(3H),1′-cyclohexane]-4′-carboxamide,

(7) trans-N-[5-(3-fluorophenyl)-2-pyrimidinyl]-3-oxospiro[5-azaisobenzofuran-1(3H), 1′-cyclohexane]-4′-carboxamide,

(8) trans-N-[5-(2-fluorophenyl)-2-pyrimidinyl]-3-oxospiro[5-azaisobenzofuran-1(3H),1′-cyclohexane]-4′-carboxamide,

(9) trans-N-[1-(3,5-difluorophenyl)4-imidazolyl]-3-oxospiro[7-azaisobenzofuran-1(3H), 1′-cyclohexane]-4′-carboxamide,

(10) trans-3-oxo-N-(1-phenyl-4-pyrazolyl)spiro[4-azaisobenzofuran-1(3H),1′-cyclohexane]-4′-carboxamide,

(11) trans-N-[1-(2-fluorophenyl)-3-pyrazolyl]-3-oxospiro[6-azaisobenzofuran-1(3H),1′-cyclohexane]-4′ -carboxamide,

(12) trans-3-oxo-N-(1-phenyl-3-pyrazolyl)spiro[6-azaisobenzofuran-1(3H),1′-cyclohexane]-4′-carboxamide,

(13) trans-3-oxo-N-(2-phenyl-1,2,3-triazol-4-yl)spiro[6-azaisobenzofuran-1(3H),1′-cyclohexane]-4′-carboxamide, and pharmaceutically acceptable salts and esters thereof.

Examples of other anti-obesity agents that can be employed in combination with a compound of Formula I and Formula VI are disclosed in “Patent focus on new anti-obesity agents,” Exp. Opin. Ther. Patents, 10: 819-831 (2000); “Novel anti-obesity drugs,” Exp. Opin. Invest. Drugs, 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000). Various pharmacological approaches for the treatment of obesity are discussed in J-A Fernandez-Lopez, Drugs: 62: 915-944 (2002) and in H. Bays, et al., “Anti-obesity drug development,” Exp. Opin. Invest. Drugs, 11: 1189-1204 (2002).

The above compounds are only illustrative of the anti-obesity agents that can be used in combination with the compounds of the present invention. As this listing of compounds is not meant to be comprehensive, the methods of the present invention may employ any anti-obesity agent, and are not limited to any particular structural class of compounds.

The combinations of the present invention are useful for the treatment or prevention of disorders associated with excessive food intake, such as obesity and obesity-related disorders. The obesity herein may be due to any cause, whether genetic or environmental.

### ASSAYS

### Cathepsin K Assay

Serial dilutions (⅓) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 ∥L of human cathepsin K (0.4 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Effect of Cathepsin K Gene Deletion on Mouse Body Weight Increase, Percentage Body Fat and Plasma Lipids After 12 Weeks on a High Fat Diet

Cathepsin K homozygous null (KO, Saftig P et al, Proc Natl Acad Sci USA. 1998; 95:13453-8) and wild-type littermate controls (WT, mixed 129/C57BL/6 background) were fed a high fat diet (HFD, 35% fat, 35% carbohydrate by weight, Bio-Serv (F3282), n=10-12/group) for 12 weeks starting at an age of age approximately 8 weeks. The animals were weighed weekly and food consumption was determined. No difference in food consumption between groups was detected. The percentage of body fat for each animal was determined using dual energy X-ray absorptiometry after 12 weeks on the HFD. At termination, blood was taken for determination of plasma cholesterol triglycerides and leptin.

| | Male WT | Male KO | Female WT | Female KO |
|---|---|---|---|---|
| Body weight gain | 14.9 ± 2.0^{a} | 12.9 ± 1.8 | 13.4 ± 2.3 | 6.8 ± 1.6** |
| after 12 weeks on | | | | |
| HFD | | | | |
| % body fat after | 32.8 ± 2.7 | 32.5 ± 1.0 | 33.7 ± 3.8 | 22.1 ± 2.8* |
| 12 weeks on HFD | | | | |
| Plasma cholesterol | 5.19 ± 0.24 | 3.65 ± 0.27** | 3.73 ± 0.27 | 3.14 ± 0.12* |
| (mM) | | | | |
| Plasma | 1.76 ± 0.11 | 1.42 ± 0.07* | 1.64 ± 0.17 | 1.59 ± 0.08 |
| triglycerides (mM) | | | | |
| Plasma leptin | 83.75 ± 14.46 | 37.25 ± 5.30* | 32.24 ± 8.73 | 13.68 ± 5.91* |
| (ng/mL) | | | | |

^{a}±SEM.
*p < 0.05,
**p < 0.01, vs WT

### Differentiation of 3T3-L1 Fibroblasts to Adipocytes in the Presence of Cathepsin K Inhibitors

3T3-L1 fibroblasts were seeded in 6-well plates, grown to confluence in regular DMEM media (containing high glucose, 10% serum, 1% antibiotic P/S, 10 mM HEPES) and maintained at confluence for 2 days. Cells were differentiated to an adipocyte phenotype using a 6-day protocol as follows. Cells were cultured in DMEM media containing insulin (5 μg/mL), IBMX (0.5 μM) and dexamethasone (0.25 μM) for 2 days, after which IBMX and dexamethasone were removed. Cells were incubated for a further 2 days in DMEM media containing insulin and then subsequently for 2 further days in DMEM media. Fat droplets (adipocytes) were usually observed after 4 days.

Cathepsin K inhibitors were tested at concentrations of 1 nM to 1 mM and were present in the various media from day 0. At day +4, fat droplets were observed in control cells, whereas cells with the cathepsin inhibitors had conserved their fibroblast phenotype.

What is claimed:

1. A method for treating obesity, treating an obesity related disorder, preventing weight gain, preventing weight regain or using for weight maintenance comprising administering a therapeutically effective amount of a cathepsin K inhibitor.

2. The method of claim 1 wherein the cathepsin K inhibitor is represented by formula I: 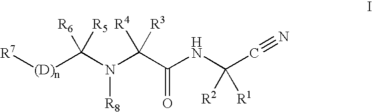
wherein
R¹ is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
R² is hydrogen, C₁₋₆ alkyl or C₂₋₆ alkenyl wherein said alkyl and alkenyl groups are optionally substituted with one to six halo, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —OR⁹, —N(R¹²)₂, aryl, heteroaryl or heterocyclyl wherein said aryl, heteroaryl and heterocyclyl groups are optionally substituted with one or two substitutents independently selected from C₁₋₆ alkyl, halo, hydroxyalkyl, hydroxy, alkoxy or keto;
or R¹ and R² can be taken together with the carbon atom to which they are attached to form a C₃₋₈ cycloalkyl or heterocyclyl ring wherein said ring system is optionally substituted with one or two substituents independently selected from C₁₋₆ alkyl, hydroxyalkyl, haloalkyl, or halo;
R³ is hydrogen, C₁₋₆ alkyl or C₂₋₆ alkenyl wherein said alkyl and alkenyl groups are optionally substituted with C₃₋₆ cycloalkyl or one to six halo;
R⁴ is hydrogen, C₁₋₆ alkyl or C₂₋₆ alkenyl wherein said alkyl and alkenyl groups are optionally substituted with C₃₋₆ cycloalkyl or one to six halo;
or R³ and R⁴ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted with one or two substitutents independently selected from C₁₋₆ alkyl, halo, hydroxyalkyl, hydroxy, alkoxy or keto;
R⁵ is selected from hydrogen or C₁₋₆ alkyl substituted with 1-6 halo;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with one, two, or three substituents independently selected from halo, C₁₋₆ alkyl, C₁₋₆ haloalkyl, C₃₋₆ cycloalkyl, haloalkoxy, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹), cyano, or aryl which is optionally substituted with —SO₂R¹²;
each D is independently C₁₋₃ alkyl, C₂₋₃ alkenyl, C₂₋₃ alkynyl, aryl, heteroaryl, C₃₋₈ cycloalkyl or heterocyclyl wherein each said aryl, heteroaryl, cycloalkyl and heterocyclyl groups, which may be monocyclic or bicyclic, is optionally substituted on either the carbon or the heteroatom with one to five substituents independently selected from C₁₋₆ alkyl, haloalkyl, halo, keto, alkoxy, —SR⁹, —SR¹², —OR⁹, —OR¹², N(R¹²)₂, —SO₂R⁹, or —SO₂R¹⁰;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR⁹, —OR¹⁰, —C(O)R¹⁰, —R¹⁰C(O)R⁹, —C(O)R⁹, —C(O)N(R^{a})(R^{b}), —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SOₘN(R^{c})(R^{d}), —SOₘCH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R⁹, —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)(R⁹), R¹⁰S—, —C(R^{a})(R^{b})NR^{a}C(R^{a})(R^{b})(R⁹), —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a}), C(R^{a})(R^{b})C(O)N(R^{a})(R^{b}), —B(OH)₂, —OCH₂O— or 4,4,5,5-tetramethyl-1,3,2-dioxaborolan-2-yl; wherein said groups are optionally substituted on either the carbon or the heteroatom with one to five substituents independently selected from C₁₋₆ alkyl, halo, keto, cyano, haloalkyl, hydroxyalkyl, —OR⁹, —NO₂, —NH₂, —NHS(O)₂R⁸, —R⁹SO₂R¹², —SO₂R¹², —SO(R¹²), —SR¹², —SR⁹, —SOₘN(R^{c})(R^{d}), —SOₘN(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —C(O)(R^{a})(R^{b}), —N(R¹⁰)C(R¹⁰)(R¹¹)(R⁹), —N(R¹⁰)CO(R⁹), —NH(CH₂)₂OH, —NHC(O)OR¹⁰, —Si(CH₃)₃, heterocycyl, aryl, or heteroaryl;
R⁸ is hydrogen or C₁₋₆ alkyl;
or R⁴ and R⁸ or can be taken together with any of the atoms to which they may be attached or are between them to form a 4-10 membered heterocyclyl ring system wherein said ring system, which may be monocyclic or bicyclic, is optionally substituted with one or two substituents independently selected from C₁₋₆ alkyl, halo, hydroxyalkyl, hydroxy, keto, —OR¹⁰, —SR¹⁰ or —N(R¹⁰)₂;
R⁹ is selected from the group consisting of hydrogen, aryl, aryl(C₁₋₄)alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with one, two, or three substituents independently selected from halo, alkoxy or —SO₂R¹²;
R¹⁰ is hydrogen or C₁₋₆ alkyl
R¹¹ is hydrogen or C₁₋₆ alkyl;
R¹² is hydrogen or C₁₋₆ alkyl which is optionally substituted with one, two, or three substituents independently selected from halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with one, two, or three substituents independently selected from C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with one or two substituents independently selected from C₁₋₆ alkyl and halo;
R^{c} is hydrogen or C₁₋₆ alkyl which is optionally substituted with one, two, or three substituents independently selected from halo or —OR⁹;
R^{d} is hydrogen or C₁₋₆ alkyl which is optionally substituted with one, two, or three substituents independently selected from halo or —OR⁹;
or R^{c} and R^{d} can be taken together with the nitrogen atom to which they are attached or are between them to form a C₃₋₈ heterocyclyl ring which is optionally substituted with one or two substituents independently selected from C₁₋₆ alkyl, halo hydroxyalkyl, hydroxy, alkoxy or keto;
n is independently selected from an integer from zero to three;
each m is independently selected from an integer from zero to two;
and the pharmaceutically acceptable salts, stereoisomers and N-oxide derivatives thereof.

3. The method of claim 2 wherein the cathepsin K inhibitor is
N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide;
N¹-(1-cyanocyclopropyl)-4-fluoro-N²-{(1S)-2,2,2-trifluoro-1-[4′-(methylsulfinyl)-1,1′-biphenyl-4-yl]ethyl}-L-leucinamide;
N¹(cyanomethyl)-N²{(1S)-2,2,2-trifluoro-1-[4′-(methylsulfonyl)-1,1′-biphenyl-4-yl]ethyl}-L-leucinamide;
N²{(1S)-1-[4′-(aminosulfonyl)-1,1′-biphenyl-4-yl]-2,2,2-trifluoroethyl}-N¹(cyanomethyl)-L-leucinamide;
N¹(1-cyanocyclopropyl)-N²-{(1S)-2,2-difluoro-1-[4′-(methylsulfonyl)biphenyl-4-yl]ethyl}-4-fluoro-L-leucinamide;
or a pharmaceutically acceptable salt thereof.

4. The method of claim 1 wherein the cathepsin K inhibitor is
N-(1-{[(cyanomethyl)amino]carbonyl}cyclohexyl)-4-(4-propylpiperazin-1-yl)benzamide;
N-(1-{[(cyanomethyl)amino]carbonyl}cyclohexyl)-4-[1-(2-methoxyethyl)piperidin-4-yl]benzamide;
or a pharmaceutically acceptable salt thereof.

5. The method of claim 1 wherein the obesity related disorder is diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, metabolic syndrome, fatty liver; cerebral infarction, cerebral thrombosis, transient ischemic attack, orthopedic disorders, arthritis deformans, lumbodynia, emmeniopathy, infertility, overeating, bulimia, elevated plasma insulin concentrations, hyperlipidemia, endometrial cancer, breast cancer, prostate cancer, colon cancer, osteoarthritis, cholelithiasis, gallstones, coronary heart disease, abnormal heart rhythms, heart arrythmias, myocardial infarction, polycystic ovarian disease, craniopharyngioma, the Prader-Willi Syndrome, Frohlich's syndrome, GH-deficient subjects, normal variant short stature, Turner's syndrome, metabolic syndrome, or acute lymphoblastic leukemia.

6. The method of claim 5 further comprising an anti-obesity agent.

7. The method of claim 6 wherein the anti-obesity agent is an anti-diabetic agent, a lipid lowering agent or an anti-hypertensive agent.

8. The method of claim 6 wherein the anti-obesity agent is PYY; PYY₃₋₃₆; a PYY agonist; 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; aminorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; topiramate; zonisamide; or a combination thereof.

9. A pharmaceutical composition comprising a cathepsin K inhibitor and an anti-obesity agent.

10. The pharmaceutical composition of claim 9 wherein the anti-obesity agent is PYY; PYY₃₋₃₆; a PYY agonist; 5HT transporter inhibitor; NE transporter inhibitor; ghrelin antagonist; H3 antagonist/inverse agonist; MCH1R antagonist; MCH2R agonist/antagonist; MC3R agonist; NPY1 antagonist; NPY4 agonist; NPY5 antagonist; leptin; leptin agonist/modulator; leptin derivatives; opioid antagonist; orexin antagonist; BRS3 agonist; 11β HSD-1 inhibitor; CCK-A agonist; CNTF; CNTF agonist/modulator; CNTF derivative; Cox-2 inhibitor; GHS agonist; 5HT2C agonist; 5HT6 antagonist; monoamine reuptake inhibitor; UCP-1, 2, and 3 activator; β3 agonist; thyroid hormone β agonist; PDE inhibitor; FAS inhibitor; DGAT1 inhibitor; DGAT2 inhibitor; ACC2 inhibitor; glucocorticoid antagonist; acyl-estrogens; lipase inhibitor; fatty acid transporter inhibitor; dicarboxylate transporter inhibitor; glucose transporter inhibitor; serotonin reuptake inhibitors; aminorex; amphechloral; amphetamine; axokine; benzphetamine; chlorphentermine; clobenzorex; cloforex; clominorex; clortermine; cyclexedrine; dextroamphetamine; diphemethoxidine, N-ethylamphetamine; fenbutrazate; fenisorex; fenproporex; fludorex; fluminorex; furfurylmethylamphetamine; levamfetamine; levophacetoperane; mefenorex; metamfepramone; methamphetamine; nalmefene; norpseudoephedrine; pentorex; phendimetrazine; phenmetrazine; phytopharm compound 57; picilorex; topiramate; zonisamide; or a combination thereof.

11. Use of a cathepsin K inhibitor in the manufacture of a medicament for treating an obesity related disorder, preventing weight regain or for weight maintenance.

12. Use according to claim 11, wherein said cathepsin K inhibitor is as defined in any one of claims 2 to 4.

13. Use according to claim 11 or 12, wherein the obesity related disorder is as defined in claim 5.

14. A cathepsin K inhibitor for use in treating an obesity related disorder, preventing weight regain or for weight maintenance.

15. A cathepsin K inhibitor according to claim 14, as defined in claim 2, 3 or 4.
